(19) United States
(12) Patent Application Publication
(10) Pub. No.: US 2020/0286626 A1
(43) Pub. Date: Sep. 10, 2020

(54) HEALTH MANAGEMENT SUPPORT DEVICE, METHOD, AND NON-TRANSITORY COMPUTER-READABLE STORAGE MEDIUM STORING PROGRAM

(71) Applicants: OMRON HEALTHCARE Co., Ltd., Kyoto (JP); OMRON Corporation, Kyoto-shi (JP)

(72) Inventors: Hiroshi USUI, Kyoto (JP); Naoki TSUCHIYA, Kyoto (JP); Yoshiyuki MORITA, Kyoto (JP); Yasushi MATSUOKA, Kyoto (JP)

(21) Appl. No.: 16/884,249

(22) Filed: May 27, 2020

Related U.S. Application Data
(63) Continuation of application No. PCT/JP2018/041716, filed on Nov. 9, 2018.

(30) Foreign Application Priority Data
Nov. 30, 2017 (JP) 2017-230603

(51) Int. Cl. G16H 50/30; G16H 50/70; G06K 9/62

(52) U.S. Cl. CPC G16H 50/30; G16H 50/70; G06K 9/6202; G06K 9/623; G06K 9/6262

(57) ABSTRACT

A health management support device according to an aspect of the present invention is capable of providing appropriate support information to a user for whom health state improvement is desired, without relying on the user's own behavior log information. A health information database (21) stores medical examination information and lifestyle information for each user. Further, a lifestyle score is calculated for each of the users on the basis of the lifestyle information and stored in the health information database (21). Then, from among the users described above, a user to whom a classification of the medical examination information is “monitoring required” is selected as a supported user, and a goal user who is to serve as an example of health state improvement for the supported user is selected on the basis of attribute information, the medical examination information, and the lifestyle score stored in the health information database (21). Then, health management support information is generated on the basis of the lifestyle information of this selected goal user and transmitted to a terminal of the supported user.

### CROSS-REFERENCE TO RELATED APPLICATIONS

This is a continuation of international Application 2017-230603, with an international filing date of Nov. 30, 2017, and PCT/JP2018/041716 with an international tiling date of Nov. 9, 2018, and filed b applicant, the disclosure of which is hereby incorporated by reference in its entirety.

### TECHNICAL FIELD

The present invention relates to a health management support device, a method, and a non-transitory computer-readable storage medium storing a program that have a function for generating information for supporting a user in health management.

### BACKGROUND ART

JP 2015-210539 A describes technology for recording, as logs, a behavior of a person and corresponding biological information of the person at times before and after the behavior and, on the basis of the measured biological information and with reference to the logs, presenting behavioral advice for improving or maintaining the biological information.

### SUMMARY OF INVENTION

However, in the invention described in JP 2015-210539 A, with reference to the user's own biological information and the user's own behavior logs, the behavioral advice is generated and personally presented to the user. Thus, in a state where the behavior logs are not recorded or are recorded but the amount of information is insufficient, beneficial behavioral advice cannot be provided.

This invention, according to one aspect, has been made with reference to the above circumstances, and an object of the present invention is to provide a health management support device, a method, and a non-transitory computer-readable storage medium storing a program capable of providing appropriate support information to a user for whom health state improvement is desired, without relying on the user's own behavior log information.

A health management support device according to an aspect of the invention includes: a storage medium configured to store, in association with each of a plurality of users, information indicating a health state of each user and information indicating a lifestyle of each user; a first selection unit configured to select, on the basis of the information indicating a health state thus stored, a first user, from among the plurality of users, to be supported in health management; a second selection unit configured to select, on the basis of the information indicating a health state thus stored and the information indicating a lifestyle thus stored, a second user, from among the plurality of users, having a health state that is to serve as a goal for the first user thus selected; a support information generation unit configured to read, from the storage medium, the information indicating a lifestyle of the second user thus selected and generate support information for supporting the health management of the first user on the basis of the information indicating a lifestyle thus read; and an information output unit configured to output the support information thus generated.

According to the configuration described above, for example, a user for whom health state improvement is desired is selected as a supported user, a goal user who is to serve as an example of health management for the supported user is selected, and the health management support information generated on the basis of the lifestyle of the goal user is provided to the supported user. Thus, it is possible to automatically provide, to the user, health management support information beneficial for the supported user. Further, it is possible for the user to work on his or her own lifestyle improvement by using the lifestyle of another person as an example.

In the health management support device according to the aspect described above, the information indicating a health state of each user may include attribute information of each user, and the second selection unit may be configured to select, on the basis of the attribute information, a user having an attribute that matches that of the first user within a predetermined range, as a candidate for the second user.

According to this configuration, when a candidate for the goal user is selected, a user having an attribute that matches that of the supported user within a predetermined range is selected as a candidate for the goal user. Thus, it is possible to provide the health management support information to the supported user on the basis of the lifestyle of a goal user having a matching attribute.

In the health management support device according to the aspect described above, the information indicating a health state of each user may include information indicating a medical examination result, and evaluation information indicating a result of evaluating the information indicating a medical examination result using a plurality of ratings on the basis of criteria defined in advance, the first selection unit may be configured to select, as the first user, a user to whom the evaluation information does not satisfy the criteria set in advance, and the second selection unit may be configured to select a candidate for the second user from among the plurality of users by comparing the evaluation information corresponding to the first user with the evaluation information corresponding to each of the plurality of users.

For example, the second selection unit is configured to select, on the basis of the evaluation information stored in the storage medium, a user to whom an evaluation result in the past is identical to an evaluation result of the first user in the present and to whom the evaluation result has improved from the evaluation result in the past during a period from the past to the present as a candidate for the second user.

According to the configuration described above, the selection of the supported user and the selection of the goal user are each performed on the basis of the evaluation information included in the medical examination result. This makes it possible to appropriately select the supported user and the goal user without requiring complex information processing. For example, a user to whom the evaluation of the medical examination result in the past is similar to that of the supported user in the present but to whom the evaluation has improved during the period from the past to the present is selected as a candidate for the goal user, and thus it is possible for the supported user to work on his or her own health state improvement with another user who has experienced the present health state of the supported user in the past and subsequently made improvements serving as the goal.

In the health management support device according to the first aspect described above, the health management support device may further include a score calculation unit configured to calculate, for each of the plurality of users, a score indicating an effort level in maintaining or improving a health state of each user on the basis of the information indicating a lifestyle of each user thus stored, and the second selection unit may be configured to select the second user from among the candidates for the second user thus selected on the basis of the score calculated for each of the candidates.

For example, the second selection unit is configured to select, on the basis of the score, a user to whom a score in the present is greater than or equal to a first threshold value and to whom an improvement amount of the score in the present relative to a score in the past is greater than or equal to a second threshold value as the second user.

According to the configuration described above, from among the candidates for the goal user, a user who has a high lifestyle score in the present and a high degree of improvement in the lifestyle score is selected as the goal user, and thus health management support information based on a more preferable lifestyle can be presented to the supported user as an example.

In the health management support device according to the aspect described above, the information indicating a health state of each user may include information indicating a medical examination result, evaluation information by a test item indicating a result of evaluating the information indicating a medical examination result using a plurality of ratings on the basis of criteria defined in advance for each test item, and comprehensive evaluation information determined on the basis of the evaluation information by a test item, the first selection unit may be configured to select, as the first user, a user to whom the comprehensive evaluation information does not satisfy comprehensive criteria set in advance, and the second selection unit may be configured to select, on the basis of the evaluation information by a test item, a user to whom an evaluation result in the past, for a specific test item, is identical to an evaluation result of the first user in the present and to whom the evaluation result has improved from the evaluation result in the past during a period from the past to the present as a candidate for the second user.

More specifically, the first selection unit is configured to select, as a supported user, a user to whom a classification of evaluation information in the present is “monitoring required”, and the second selection unit is configured to select, as a goal user, a user having a gender and an age demographic matching those of the supported user, to whom a classification of evaluation information in the past matches the classification of “monitoring required” in the present of the supported user thus selected, and to whom the classification of evaluation information in the present has improved to “normal” or “substantially normal”.

According to the configuration described above, by test item, a user to whom the evaluation result in the past is the same as the evaluation result of the supported user in the present and to whom the evaluation result has improved from the evaluation result in the past during the period from the past to the present is selected as a candidate for the goal user. Thus, it is possible for the supported user to work on his or her own health state improvement with another user who experienced improvement on a test item formerly evaluated as improvement desired serving as the goal.

According to the invention, it is possible to provide a health management support device, a method, and a non-transitory computer-readable storage medium storing a program capable of providing appropriate support information to a user for whom health state improvement is desired, without relying on the user's own behavior log information.

### BRIEF DESCRIPTION OF DRAWINGS

FIG. 1 is a functional block diagram illustrating one application example of a health management support device according to an embodiment of the invention.

FIG. 2 is a diagram illustrating an overall configuration of a system by explaining a first example of the health management support device according to an embodiment of the invention.

FIG. 3 is a block diagram illustrating a functional configuration of the health management support device by explaining the first example of the health management support device according to an embodiment of the invention.

FIG. 4 is a flowchart illustrating a processing procedure and processing content of health management support control by the health management support device illustrated in FIG. 3.

FIG. 5 is a flowchart illustrating a processing procedure and processing content of goal user selection, from among the steps of the health management support control illustrated in FIG. 4.

FIG. 6 is a diagram illustrating an example of information stored in a health information database provided by the health management support device illustrated in FIG. 3.

FIG. 7 is a diagram illustrating an example of medical examination information by explaining a second example of the health management support device according to an embodiment of the invention.

### DESCRIPTION OF EMBODIMENTS

An embodiment according to an aspect of the invention (hereinafter, also referred to as “an embodiment”) will be described below with reference to the drawings. However, the embodiment described below is merely illustrative of the invention in all respects. Of course, various modifications and variations can be made without departing from the scope of the invention. That is, specific configurations according to the embodiment may be adopted as appropriate in the implementation of the invention.

### Application Example

First, an example of a scenario to which the invention is applied will be described.

### Configuration

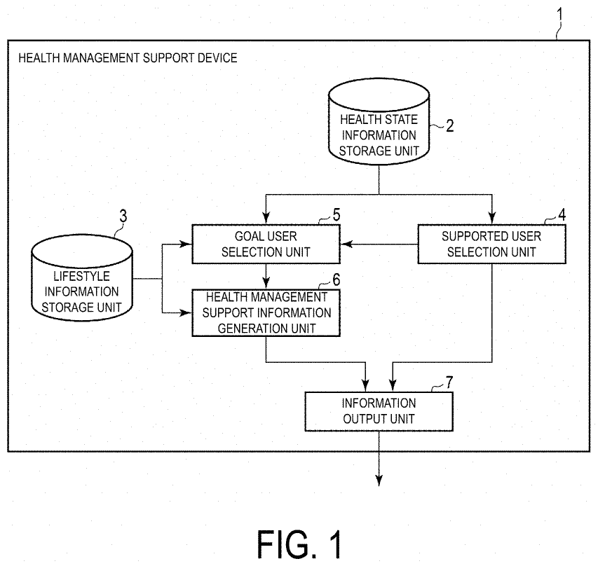

FIG. 1 schematically illustrates a functional configuration of a health management support device according to this application example. This health management support device 1 is realized by a server computer operated by an operator providing, for example, a health management support service.

The health management support device 1 includes a health state information storage unit 2, a lifestyle information storage unit 3, a supported user selection unit 4 as a first selection unit, a goal user selection unit 5 as a second selection unit, a health management support information generation unit 6, and an information output unit 7.

The health state information storage unit 2 is used to store information indicating a health state of each of a plurality of users. The information indicating a health state is composed of, for example, information indicating a medical examination result and includes: attribute information of the user, data indicating a medical examination result, and evaluation information (a classification) obtained by evaluating the data using a plurality of ratings on the basis of a reference value defined in advance. The attribute information of the user includes at least an age and a gender but may also include other information indicating an occupation, a place of residence, a medical history, and the like. The classification of the evaluation information is defined by a medical institution that performs a medical examination, a health insurance union, or a local government and is indicated by, for example, eight ratings on a scale of A to H, five ratings on a scale of 1 to 5, and four ratings on a scale of A to D. Note that the information indicating a medical examination result can be collected from, for example, a database of a medical institution that performs medical examinations or of an operator who manages information indicating the medical examination results obtained from the medical institution.

The lifestyle information storage unit 3 is used to store information indicating a lifestyle of each of the plurality of users. The information indicating a lifestyle includes information indicating a plurality of elements in the daily life of the user, such as food intake, sleep, and exercise, for example. Specifically, “food intake” may be indicated by frequency, time period, menu content, and the like, “sleep” may be indicated by sleep time, and “exercise” may be indicated by the amount of activity. The information indicating a lifestyle is expressed as, for example, a score of the measured value for each element described above.

The supported user selection unit 4 performs processing in which the classification included in the health state information stored in the health state information storage unit 2 is compared to a reference set in advance, and a user whose classification does not satisfy the reference and for whom health state improvement is desired is selected as a supported user.

The goal user selection unit 5 performs processing in which a user who is to serve as a goal is selected when the health state of the selected supported user is to be improved, on the basis of the health state information stored in the health state information storage unit 2 and the lifestyle information stored in the lifestyle information storage unit 3. The selection of this goal user is made by using, for example, the attribute information of the user included in the health state information, the classification of the medical examination result, and a lifestyle score as selection conditions. Specifically, a user, whose attribute information matches that of the supported user within a predetermined range, whose classification of the medical examination result is greater than or equal to a reference classification determined as normal, and whose lifestyle score is greater than or equal to a threshold value, is selected as the goal user. Note that the lifestyle score indicates an effort level in maintaining or improving the health state of the user and can be calculated on the basis of the lifestyle information for each user, for example.

The health management support information generation unit 6 performs processing in which the lifestyle information of the selected goal user is used to generate health management support information appropriate for the supported user described above. The health management support information includes, for example, the content of the lifestyle of the goal user and advice information based on this content.

The information output unit 7 performs processing in which the generated health management support information is transmitted to a terminal of the supported user by email or the like. The destination address can be obtained by, for example, using the attribute information included in the information indicating a health state of the user.

### Operation

The health management support device 1 operates as described below, for example.

That is, it is assumed that information indicating group medical examination results for an employee group at a certain office is transmitted from a server of a medical institution to the health management support device 1 and stored in the health state information storage unit 2. In this case, first, the supported user selection unit 4 selects, on the basis of the stored information indicating a medical examination result, an employee whose classification is, for example, “monitoring required” as the supported user.

Next, the goal user selection unit 5 selects, on the basis of the information indicating a medical examination result stored in the health state information storage unit 2, employees having a gender and an age demographic matching those of the supported user, whose classification of a medical examination result in the past corresponds to the classification (“monitoring required”) of the supported user in the present, and whose classification in the present has improved to “normal” or “substantially normal” as candidates for the goal user, for example. Then, from among the candidates for the goal user, a user, having a lifestyle score in the present that is greater than or equal to a first threshold value and having a lifestyle score in the present that has improved from a lifestyle score in the past by a second threshold value or greater, is determined to be the goal user.

Next, in the health management support information generation unit 6, health management support information is generated from, for example, the lifestyle information of the employee in the present determined to be the goal user; and the advice information, and this generated health management support information is transmitted by the information output unit 7 to the terminal of the supported user using email or the like.

### Effect

Thus, according to the health management support device 1 described above, from within the employee group who received a group medical examination, an employee whose medical examination result is determined to be “monitoring required”, for example, is selected as a supported user in health management. Further, from within the employee group, employees having a gender and an age demographic matching those of the selected supported user, whose classification of a medical examination result in the past corresponds to the classification (“monitoring required”) of the supported user in the present, and whose classification in the present has improved to “normal” or “substantially normal” are selected as candidates for the goal user. Then, from among the selected candidates for the goal user, a user having a lifestyle score in the present that is greater than or equal to the first threshold value and having a lifestyle score in the present that has improved from a lifestyle score in the past by the second threshold value or greater is determined to be the goal user. Then, support information generated on the basis of the lifestyle of the goal user is presented to the supported user as a lifestyle example.

As a result, it is possible to present the lifestyle of another person who has a history of improving the evaluation of the medical examination result, as an example, to a user whose medical examination result is determined to be “monitoring required”. Thus, it is possible for a user determined to be “monitoring required” to work on his or her own lifestyle improvement by referring to the lifestyle of another person serving as the example.

Moreover, in the selection of the goal user described above, employees having a gender and an age demographic matching those of the supported user and to whom a similar determination of a medical examination result has been made in the past but who has improved in the present are selected as candidates for the goal user, and thus users whose situation is similar to that of the supported user can be selected as candidates for the goal user.

Furthermore, from among the candidates for the goal user, a user having a high lifestyle score in the present and a high degree of improvement in the lifestyle score is selected as the goal user, and thus a more preferable lifestyle can be presented to the supported user as an example.

### First Example

### Configuration

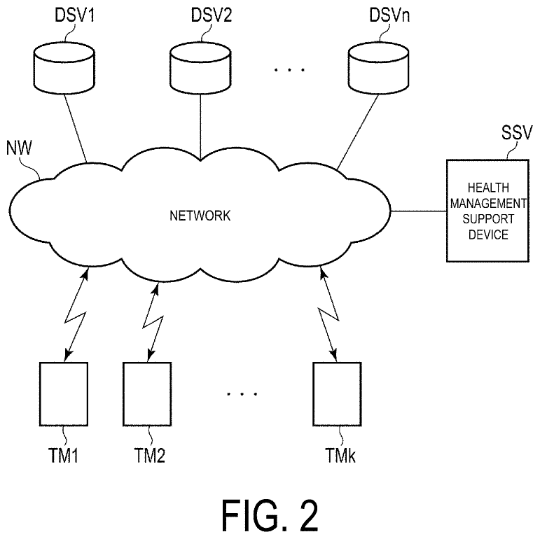

FIG. 2 is a schematic configuration diagram of a system including the health management support device according to a first example of the invention.

This system is configured to be capable of connecting a plurality of medical information servers DSV1 to DSVn and a plurality of user terminals TM1 to TMk to a health management support device SSV via a network NW.

The medical information servers DSV1 to DSVn are each composed of, for example, a data server of a medical institution that performs medical examinations or of an operator who manages the information indicating a medical examination result obtained from the medical institution and transmits the information indicating a medical examination result to the health management support device SSV via the network NW. The information indicating a medical examination result will be described later.

The user terminals TM1 to TMk include at least one of: a wearable terminal including a sensor; and a mobile information terminal such as a smartphone, for example. Moreover, the user terminals TM1 to TMk have a function for transmitting: measurement data pertaining to the lifestyle of the user measured by the plurality of sensors; or report data of the user's own lifestyle manually input by the user himself/herself to the health management support device SSV via the network NW.

The measurement data pertaining to the lifestyle includes, for example, food intake data, exercise data, and sleep data. The food intake data includes, for example, data indicating a menu, a food intake time, a food intake frequency, and the like. The exercise data is indicated by, for example, a step count, a moved distance, and an amount of activity. The sleep data includes, for example, data indicating sleep time. Note that the food intake data may include an alcohol intake amount, an alcohol intake frequency per week, and the like.

Further, each of the user terminals TM1 to TMk also has a function for: receiving the health management support information addressed to its own terminal and transmitted from the health management support device SSV by e-mail or the like, storing the support information, and displaying the information on a display.

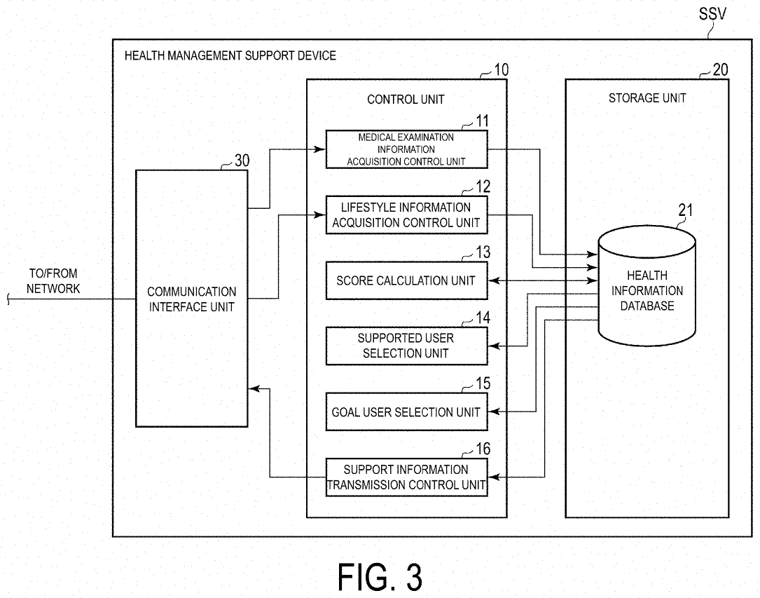

The health management support device SSV includes a cloud or a server computer operated by the operator providing the health management support service and is configured as described below, for example. FIG. 3 is a block diagram illustrating a functional configuration thereof.

The health management support device SSV includes a control unit 10, a storage unit 20, and a communication interface unit 30. The communication interface unit 30 transmits and receives data to and from the medical information servers DSV1 to DSVn and the user terminals TM1 to TMk via the network NW.

The storage unit 20 includes a health information database 21 as a database necessary for implementing this example, using a non-volatile memory capable of writing and reading as needed, such as a hard disk drive (HDD) or a solid state drive (SSD); and a volatile memory such as dynamic random-access memory (DRAM).

The health information database 21 is used to store, in association with identification information of the user (user ID), attribute information, information indicating a medical examination result (medical examination information), information indicating a lifestyle (lifestyle information), and information obtained by scoring the lifestyle (lifestyle score). This information is updated to the most recent information each time a medical examination is performed, but the information obtained from the previous medical examination remains without being deleted.

The attribute information includes, for example, the gender and the age of the user himself/herself. Note that the attribute information of the user may also include other information indicating an occupation, a place of residence, a medical history, and the like. Further, the attribute information also includes the name, a contact phone number, and email address information of the user.

The medical examination information includes test data indicating a test value for each test item, and a classification indicating a comprehensive evaluation result of the test data. The classification is obtained by evaluating the test data using a plurality of ratings on the basis of a reference value, and the reference value is set, for example, on the basis of the guideline of the Japan Society of Ningen Dock. The classifications differ according to the medical institution that performs the medical examination, the health insurance union, or the local government and are indicated by, for example, eight ratings on a scale of A to H, five ratings on a scale of 1 to 5, and four ratings on a scale of A to D.

The lifestyle information is information transmitted from the user terminals TM1 to TMk and includes information indicating, for example, the daily food intake, sleep, exercise, and the like of the user, as previously described. Specifically, “food intake” is indicated by frequency, time period, menu content, and the like, “sleep” is indicated by sleep time, and “exercise” is indicated by the amount of activity.

The lifestyle score indicates an effort level in maintaining or improving the health state of the user and is calculated on the basis of the lifestyle information for each user, for example. The calculation method of this lifestyle score will be described later.

The control unit 10 includes a central processing unit (CPU) and a working memory as hardware and is provided with, as control functions necessary for implementing the first example, a medical examination information acquisition control unit 11, a lifestyle information acquisition control unit 12, a score calculation unit 13, a supported user selection unit 14, a goal user selection unit 15, and a support information transmission control unit 16. These control function units are all realized by causing the CPU to execute a program stored in a program memory (not illustrated).

The medical examination information acquisition control unit 11 receives the medical examination information (including the classification) of the user transmitted from the medical information servers DSV1 to DSVn via the communication interface unit 30. Then, processing is performed in which the medical examination information is stored in association with the user ID in the health information database 21. Note that the method for acquiring the health information may be any of a push method in which medical examination information is received from the medical information servers DSV1 to DSVn each time health information is transmitted; and a polling method in which a transmission request is transmitted to the medical information servers DSV1 to DSVn to acquire the medical examination information.

The lifestyle information acquisition control unit transmits a transmission request for lifestyle information to the corresponding user terminals TM1 to TMk in response to the acquisition of the medical examination information described above and in response to the request, receives the lifestyle information transmitted from the user terminals TM1 to TMk via the communication interface unit 30, for example. Then, processing is performed in which the lifestyle information is stored in the health information database 21 in association with the user ID.

The score calculation unit 13 calculates the lifestyle score indicating the effort level in maintaining and improving the health state for each user, on the basis of the lifestyle information stored in the health information database 21. Then, processing is performed in which the calculated lifestyle score is stored in the health information database 21 in association with the user ID.

Processing is then performed in which the supported user selection unit 14 selects, from among a plurality of users whose health information is stored in the health information database 21, a user whose classification of the medical examination information is, for example, “monitoring required” as the supported user in health management.

The goal user selection unit 15 has the processing functions described below.

(1) Selecting, on the basis of the attribute information stored in the health information database 21, a user having a gender and an age demographic matching those of the supported user selected by the supported user selection unit 14.

(2) Selecting, with reference to the medical examination information stored in the health information database 21, users whose previous classification of the test data corresponds to the current classification (“monitoring required”) of the supported user and whose current classification has improved to “normal” or “substantially normal” as goal user candidates.

(3) Determining, with reference to the lifestyle scores stored in the health information database 21, a user, from among the selected goal user candidates, having a current lifestyle score greater than or equal to the first threshold value and having a current lifestyle score that has improved from the previous lifestyle score by the second threshold value or greater as the goal user.

The support information transmission control unit 16 reads the lifestyle information of the goal user thus determined from the health information database 21 and generates the health management support information with an advice message added to this lifestyle information. Then, processing is performed in which the address information included in the attribute information of the supported user is used to transmit the generated health management support information to the terminal of the supported user.

### Operation

Next, the operation of the health management support device SSV configured as described above will be described.

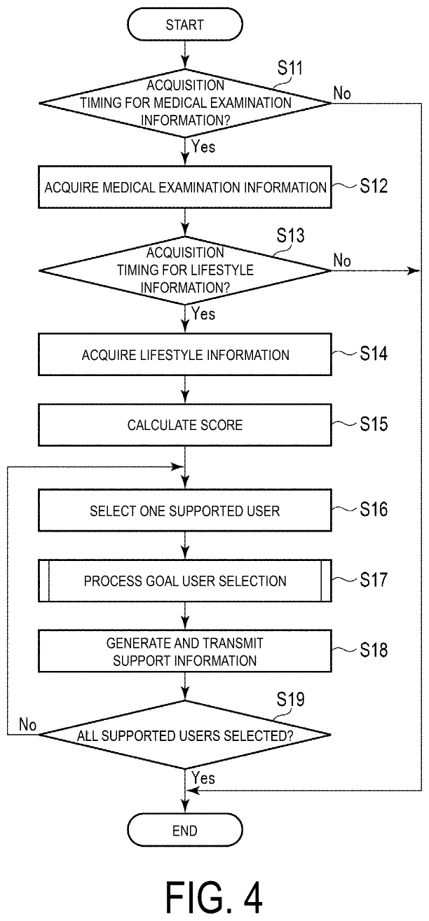

FIG. 4 is a flowchart illustrating a processing procedure and processing content of health management support control by the health management support device SSV.

### (1) Acquisition of Medical Examination Information

For example, in a medical institution, when a medical examination is performed for each user, the test data obtained by this medical examination and the classification indicating a comprehensive evaluation determined by a physician in response to the test data are temporarily stored in the medical information servers DSV1 to DSVn as medical examination information.

In step S11, under the control of the medical examination information acquisition control unit 11, at the acquisition timing of the medical examination information, the health management support device SSV transmits a transmission request to the medical information servers DSV1 to DSVn, requesting the transmission of the medical examination information all at once or sequentially. On the other hand, when the medical information servers DSV1 to DSVn each receive the transmission request addressed to its own server, the medical information servers DSV1 to DSVn transmit, to the requesting health management support device SSV, the medical examination information of each user that not yet transmitted but has been stored during the period from the reception of the previous transmission request to the reception of the current transmission request.

The medical examination information acquisition control unit 11 of the health management support device SSV, in step S12, imports the medical examination information transmitted from the medical information servers DSV1 to DSVn via the communication interface unit 30 and stores this medical examination information in the health information database 21 in association with the user IDs. Note that, at this time, the previously received medical examination information remains in the health information database 21 without being deleted.

Thereafter, similarly, the medical examination information acquisition control unit 11 of the health management support device SSV transmits a transmission request to the medical information servers DSV1 to DSVn at each acquisition timing of the medical examination information, acquires the medical examination information not yet acquired, and stores the information in the health information database 21.

### (2) Acquisition of Lifestyle Information

In each of the user terminals TM1 to TMk, food intake data, exercise data, and sleep data, for example, are generated as information pertaining to the lifestyle of the user and stored in the memory in the terminals. The food intake data includes, for example, menu data, food intake time, food intake frequency, and the like, and the menu data can be acquired by downloading recipes from a cooking site or by capturing an image of the actual food cooked or ingredients with a camera. The food intake time and food intake frequency may be identified by a download time and a download frequency of the menu data described above in one day but may be manually input by the user.

The exercise data is indicated by, for example, a step count, a moved distance, or the amount of activity, and these can be measured by an acceleration sensor, a position sensor, an activity meter, or the like mounted on a wearable terminal or a smartphone. The sleep data includes, for example, data indicating sleep time, and this sleep time can be obtained by a light sensor and acceleration data.

In step S13, under the control of the lifestyle information acquisition control unit 12, at the acquisition timing of the lifestyle information, the health management support device SSV transmits a transmission request to the user terminals TM1 to TMk, requesting the transmission of the lifestyle information all at once or sequentially. On the other hand, upon receiving the transmission request addressed to its own terminal, each of the user terminals TM1 to TMk transmits the most recently generated lifestyle information to the requesting health management support device SSV.

The lifestyle information acquisition control unit 12 of the health management support device SSV, in step S14, imports the lifestyle information transmitted from the user terminals TM1 to TMk via the communication interface unit 30 and stores this lifestyle information in the health information database 21 in association with the user IDs. Note that, at this time, the previously received lifestyle information remains in the health information database 21 without being deleted.

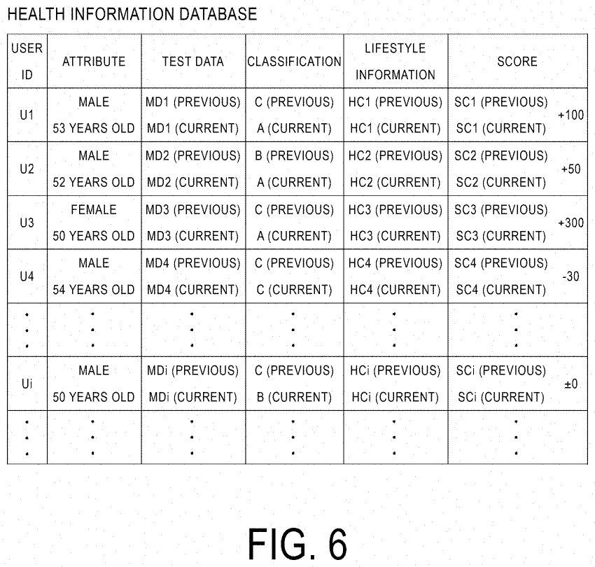

FIG. 6 illustrates an example of the attribute information, the medical examination information (test data MD1, MD2, . . . , and classification), and lifestyle information HC1, HC2, . . . of users U1, U2, . . . , acquired as described above. Note that while FIG. 6 illustrates a case where gender and age are acquired as the attribute information, an occupation, a place of residence, a medical history, and the like may be included. The attribute information can be extracted from the medical examination information but may be information manually input by the users with the user terminals TM1 to TMk.

### (3) Calculation of Lifestyle Score

Once the lifestyle information is updated in the health information database 21, in step S15, the health management support device SSV calculates, under the control of the score calculation unit 13, a score of the lifestyle on the basis of the updated lifestyle information.

For example, given α, β, and γ as the scores of food intake data, exercise data, and sleep data, respectively, a lifestyle score Tscore can be calculated as:

Tscore=w₁α+w₂β+w₃β. Note that w₁, w₂, and w₃ are weighting factors and are determined according to degrees of impact that food intake, exercise, and sleep have on lifestyle, respectively.

The score α of the food intake data can be calculated by, for example, estimating calories and salt content from the menu data and multiplying the estimated values of the calories and salt content each by a factor corresponding to the food intake time and food intake frequency in one day. The score β of the exercise data can be calculated by, for example, multiplying the step count, the moved distance, and the amount of activity each by a factor and finding the sum total. The score γ of the sleep data can be calculated by multiplying the sleep time by a constant factor.

Not that, in the calculation of the lifestyle score Tscore, a body mass index (BMI), a blood pressure value, and the like included in the medical examination information may be taken into account in addition to the food intake data, the exercise data, and the sleep data.

The score calculation unit 13 stores the lifestyle score Tscore calculated as described above in the health information database 21 in association with the user IDs. In the example of FIG. 6, SC1, SC2, . . . corresponds to such storage. Note that, in the storage area of the lifestyle score of the health information database 21, an increase or a decrease in the current lifestyle score relative to the previous lifestyle score is stored together with the score. This increase or decrease is indicative of a degree of lifestyle improvement and is later used as one indicator when selecting a goal user.

### (4) Selection of Supported User

Next, in step S16, the health management support device SSV selects, under the control of the supported user selection unit 14, one user who needs health state improvement, from among the plurality of users whose health information is stored in the health information database 21.

For example, the classification included in the health information for each user is read from the health information database 21, and a user to whom this classification is “C=Monitoring required” is selected. Note that the classification is not limited to “C=Monitoring required”, and a user to whom the classification is “D=Medical care required” or “F=Continued treatment required” may be selected.

### (5) Selection of Goal User

Then, in step S17, the health management support device SSV performs processing in which, under the control of the goal user selection unit 15, a user who is to serve as a health state improvement goal for the selected supported user is selected from among the plurality of users whose lifestyle information is stored in the health information database 21.

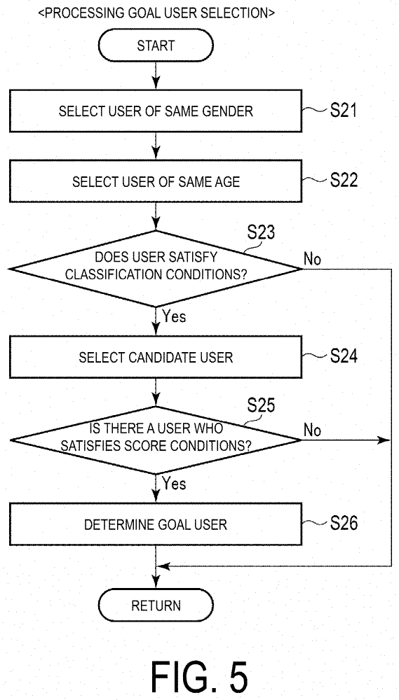

FIG. 5 is a flowchart illustrating a processing procedure and processing content of the goal user selection described above.

The goal user selection unit 15 first selects, in step S21, with reference to the attribute information stored in the health information database 21, users having a gender matching that of the supported user selected in step S16. Then, in step S22, from among the users having the same gender selected in step S21 described above, users having a matching age demographic are selected. As a result, for example, when the supported user is a “male” and “54 years old”, users who are “male” and in their “50s” are selected from among the users registered in the health information database 21 as candidates for the goal user. Note that the age demographic to be selected may be in 5-year increments rather than 10-year increments and may be limited to users who are the same age or one year older or younger than the supported user.

Next, the goal user selection unit 15 determines, in step S23, whether or not there exist users satisfying the conditions of the classification, among the selected goal user candidates having the same gender and age demographic. Then, when users corresponding to the conditions exist, in step S24, the goal user candidates are narrowed down to users satisfying the conditions of the classification.

For example, with reference to the medical examination information stored in the health information database 21, the goal user candidates are narrowed down to users whose previous classification corresponds to the current classification (“C=Monitoring required”) of the supported user and whose current classification has improved to “A=Normal” or “B=Substantially normal”.

As a result, given that the information pertaining to each user shown in FIG. 6 is stored in the health information database 21, for example, from among “male” users U1, U2, U4, Ui in their “50s”, the users U1, Ui whose previous classification is “C=Monitoring required” and whose current classification has been improved to “A=Normal” or “B=Substantially normal” are selected as the goal user candidates.

The goal user selection unit 15 further determines, in step S25, whether or not there exists a user satisfying the conditions of the lifestyle score among the goal user candidates U1, Ui selected above and, if such a user exists, determines the user to be the goal user in step S26.

For example, with reference to the lifestyle scores stored in the health information database 21, from among the goal user candidates U1, Ui selected in step S24 above, the user whose current lifestyle score is greater than or equal to the first threshold value and whose current lifestyle score has improved from the previous lifestyle score by the second threshold value is determined to be the goal user.

Using FIG. 6 as an example, given that the current lifestyle scores of the goal user candidates U1, Ui are greater than or equal to the first threshold value, the user U1 to whom an increase or decrease in the lifestyle score is the second threshold value (=50, for example) or greater is determined to be the goal user. Note that, when there exist a plurality of users satisfying the conditions of the lifestyle score, a user having the highest lifestyle score or a preset number of users in an upper level are selected from these users.

### (6) Transmission of Health Management Support Information

In step S18, the health management support device SSV then reads, under the control of the support information transmission control unit 16, the lifestyle information of the goal user thus determined from the health information database 21 and generates the health management support information with an advice message added to this lifestyle information. Then, the address information included in the attribute information of the supported user is used to transmit the generated health management support information to the terminal of the supported user.

For example, in the example of FIG. 6, lifestyle information HC1 (current) of the user U1 is read, and an improvement advice message for the lifestyle of the supported user is added to the lifestyle information HC1 (current). Then, the health management support information is passed and transmitted to the terminal of the supported user.

Note that the content of the improvement advice message is determined by, for example, comparing the lifestyle score of the goal user and the lifestyle score of the supported user for each component, such as food intake, exercise, and sleep, for example, identifying an element having a difference greater than or equal to a predetermined value, and making the message corresponding to the element. The message can be realized by, for example, selecting an applicable message from a message database (not illustrated) in the storage unit 20.

When the transmission of the support information corresponding to one supported user ends, the health management support device SSV, in step S19, determines whether or not the selection of all users who need health state improvement has been completed. Then, when, as a result of this determination, supported users who have not been selected remain, the flow returns to step S16 to select the next supported user who has not been selected, and the processing according to (5) to (6) described above is repeatedly executed. Then, when the selection of all users who need health state improvement has been completed, the series of processing is ended.

### Effect

As described in greater detail above, in the first example, in the health management support device SSV, the medical examination information of users is acquired from the medical information servers DSV1 to DSVn and stored in the health information database 21, the lifestyle information of the users is acquired from the user terminals TM1 to TMk and stored in the health information database 21, and the lifestyle score indicating the effort level in maintaining and improving the health state is calculated on the basis of the lifestyle information and stored for each user in the health information database 21. In this state, from among the plurality of users whose health information is stored in the health information database 21, a user whose classification of the medical examination information is “monitoring required”, for example, is selected as a supported user in health management, and a goal user who is to serve as an example of health state improvement for the supported user is selected on the basis of the attribute information, the medical examination information, and the lifestyle score stored in the health information database 21. Then, the health management support information is generated on the basis of the lifestyle information of this selected goal user and transmitted to the terminal of the supported user.

Thus, according to the first example, it is possible to present the lifestyle of another user who has a history of improving the evaluation of the medical examination result, as an example, to a user whose medical examination result is determined to be “monitoring required”. Thus, it is possible for a user determined to be “monitoring required” to work on his or her own lifestyle improvement by using the lifestyle of another user as the example.

Moreover, in the selection of the goal user described above, users having a gender and an age demographic matching those of the supported user and to whom a similar evaluation was made for a previous medical examination result but to whom the current evaluation has improved are selected as candidates for the goal user, and thus users whose situation is similar to that of the supported user can be selected as candidates for the goal user.

Furthermore, from among the candidates for the goal user, a user having a high lifestyle score in the present and a high degree of improvement in the lifestyle score is selected as the goal user, and thus a more preferable lifestyle can be presented to the supported user as an example.

### Second Example

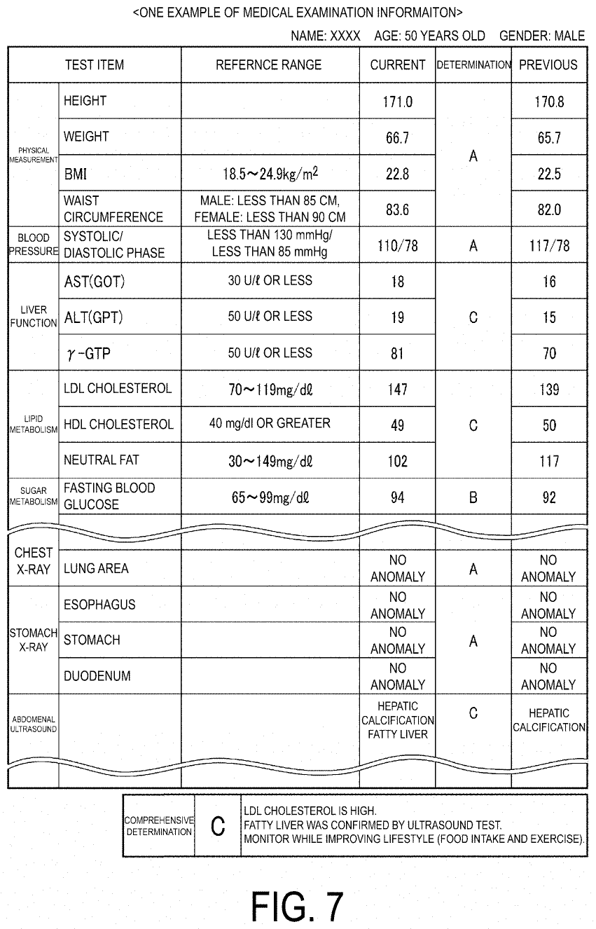

In the first example, an example has been described of a case where the classification of the medical examination information is a comprehensive determination result obtained by comprehensively evaluating all test items. However, in medical examination information, as illustrated in FIG. 7, apart from the comprehensive classification, for example, a plurality of test items may be grouped together by function, and an evaluation may be conducted for each of these functions to determine the classification.

Here, when the supported user is selected, the user may be selected on the basis of the comprehensive classification of the medical examination information and, on the other hand, when the goal user is selected, with reference to the classification by the function of the test item, users whose classification of a function of a specific test item, such as “liver function” or “lipid metabolism”, in the past is the same as the current classification of the supported user and whose current classification has improved from the previous classification may be selected as candidates for the goal user.

In this way, by the function of the test item, the users whose previous classification is the same as the current classification of the supported user and whose current classification has improved from the previous classification may be selected as candidates for the goal user. Thus, it is possible for the supported user to work on his or her own health state improvement with another user who experienced improvement on a test item formerly evaluated as improvement desired serving as the goal.

Note that while, in the example of FIG. 7, a case where test data is evaluated by the function of the test item and the classification is set is described as an example, by using medical examination information obtained by evaluating the test data by test item and setting a classification for each, it is possible to provide support information that is even more beneficial to the supported user.

### OTHER EXAMPLES

In each of the examples described above, description is made using, as an example, a case where the selections of the supported user and the goal user are made by utilizing classifications of the medical examination information obtained by an evaluation based on eight ratings on a scale of A to H used in a general hospital or clinic. However, the selections of the supported user and the goal user may be made by utilizing the classifications of a five-rating evaluation on a scale of 1 to 5 and the classifications of a four-rating evaluation on a scale of A to D adopted by a health insurance union and the managing local government.

Further, in each of the examples described above, description is made of a case where the health management support device SSV is a device independent of the medical information servers DSV1 to DSVn. However, the function of the health management support device SSV may be provided to any of the medical information servers DSV1 to DSVn.

Furthermore, in each of the examples described above, the attribute information of the user is used as one selection condition of the goal user. However, the attribute information may not necessarily be used. In addition, the respective selection conditions of the supported user and the goal user, the content of the support information, the configuration of the health information database, and the like can be variously modified in a range without departing from the gist of the invention.

In short, the invention is not limited to the embodiments described above and can be embodied by modifying the components in an implementation stage in a range without departing from the gist thereof. Further, various inventions can be formed by appropriate combinations of the plurality of components disclosed in the embodiments described above. For example, from among all components illustrated in the embodiments, several components may be deleted. Furthermore, components of different embodiments may be combined as appropriate.

The whole or part of the embodiments described above can be described as, but not limited to, the following supplementary notes.

### Supplementary Note 1

A health management support device includes

a hardware processor; and

a memory connected to the hardware processor,

the memory including

a storage medium configured to store, in association with each of a plurality of users, information indicating a health state of each user and information indicating a lifestyle of each user, and

the hardware processor being configured to

select, on the basis of the information indicating a health state thus stored, a first user, from among the plurality of users, to be supported in health management,

select, on the basis of the information indicating a health state thus stored and the information indicating a lifestyle thus stored, a second user, from among the plurality of users, having a health state that is to serve as a goal for the first user thus selected,

reading, from the storage medium, the information indicating a lifestyle of the second user thus selected and generating support information for supporting the health management of the first user on the basis of the information indicating a lifestyle thus read, and

outputting the support information thus generated.

### Supplementary Note 2

A health management support method executed by a health management support device including: a memory configured to store, in association with each of a plurality of users, information indicating a health state of each user and information indicating a lifestyle of each user; and at least one hardware processor, the health management support method including the steps of:

selecting, by using the at least one hardware processor and the memory, on the basis of the information indicating a health state thus stored, a first user, from among the plurality of users, to be supported in health;

selecting, by using the at least one hardware processor and the memory, on the basis of the information indicating a health state thus stored and the information indicating a lifestyle thus stored, a second user, from among the plurality of users, having a health state that is to serve as a goal for the first user thus selected;

reading, by using the at least one hardware processor and the memory, from the storage medium, the information indicating a lifestyle of the second user thus selected and generating support information for supporting the health of the first user on the basis of the information indicating a lifestyle thus read; and

outputting, by using the at least one hardware processor and the memory, the support information thus generated.

### Supplementary Note 3

A health management support device (1, SSV) including:

a storage medium (2, 3) configured to store, in association with each of a plurality of users, information indicating a health state of each user and information indicating a lifestyle of each user;

a first selection unit (4) configured to select, on the basis of the information indicating a health state thus stored, a first user, from among the plurality of users, to be supported in health management;

a second selection unit (5) configured to select, on the basis of the information indicating a health state thus stored and the information indicating a lifestyle thus stored, a second user, from among the plurality of users, having a health state that is to serve as a goal for the first user thus selected;

a support information generation unit (6) configured to read, from the storage medium, the information indicating a lifestyle of the second user thus selected and generate support information for supporting the health management of the first user on the basis of the information indicating a lifestyle thus read; and

an information output unit (7) configured to output the support information thus generated.

### REFERENCE SIGNS LIST

1, SSV Health management support device
2 Health state information storage unit
3 Lifestyle information storage unit
4 Supported user selection unit
5 Goal user selection unit
6 Health management support information generation unit
7 Information output unit
DSV1 to DSVn Medical information server
TM1 to TMk User terminal
NW Network
10 Control unit
11 Medical examination information acquisition control unit
12 Lifestyle information acquisition control unit
13 Score calculation unit
14 Supported user selection unit
15 Goal user selection unit
16 Support information transmission control unit
20 Storage unit
21 Health information database
30 Communication interface unit

1. A health management support device comprising:
a storage medium configured to store, in association with each of a plurality of users, information indicating a health state of each user including information indicating a medical examination result, and information indicating a lifestyle of each user;
a first selection unit configured to select, on the basis of the information indicating a health state thus stored, a first user, from among the plurality of users, to be supported in health management;
a second selection unit configured to select, on the basis of the information indicating a health state thus stored and the information indicating a lifestyle thus stored, a second user, from among the plurality of users, having a health state that is to serve as a goal for the first user thus selected;
a support information generation unit configured to read, from the storage medium, the information indicating a lifestyle of the second user thus selected and generate support information for supporting the health management of the first user on the basis of the information indicating a lifestyle thus read; and
an information output unit configured to output the support information thus generated.

2. The health management support device according to claim 1, wherein
the information indicating a health state of each user further includes attribute information of each user, and
the second selection unit is configured to select, on the basis of the attribute information, a user having an attribute that matches that of the first user within a predetermined range, as a candidate for the second user.

3. The health management support device according to claim 1, wherein
the information indicating a health state of each user further includes evaluation information indicating a result of evaluating the information indicating a medical examination result using a plurality of ratings on the basis of criteria defined in advance,
the first selection unit is configured to select, as the first user, a user to whom the evaluation information does not satisfy the criteria set in advance, and
the second selection unit is configured to select a candidate for the second user from among the plurality of users by comparing the evaluation information corresponding to the first user with the evaluation information corresponding to each of the plurality of users.

4. The health management support device according to claim 3, wherein
the second selection unit is configured to select, on the basis of the evaluation information stored in the storage medium, a user to whom an evaluation result in the past is identical to an evaluation result of the first user in the present and to whom the evaluation result has improved from the evaluation result in the past during a period from the past to the present as a candidate for the second user.

5. The health management support device according to claim 2, further comprising
a score calculation unit configured to calculate, for each of the plurality of users, a score indicating an effort level in maintaining or improving a health state of each user on the basis of the information indicating a lifestyle of each user thus stored, wherein
the second selection unit is configured to select the second user from among the candidates for the second user thus selected on the basis of the score calculated for each of the candidates.

6. The health management support device according to claim 5, wherein
the second selection unit is configured to select, on the basis of the score, a user to whom a score in the present is greater than or equal to a first threshold value and to whom an improvement amount of the score in the present relative to a score in the past is greater than or equal to a second threshold value as the second user.

7. The health management support device according to claim 1, wherein
the information indicating a health state of each user includes
information indicating a medical examination result,
evaluation information by a test item indicating a result of evaluating the information indicating a medical examination result using a plurality of ratings on the basis of criteria defined in advance for each test item, and
comprehensive evaluation information determined on the basis of the evaluation information by a test item,
the first selection unit is configured to select, as the first user, a user to whom the comprehensive evaluation information does not satisfy comprehensive criteria set in advance, and
the second selection unit is configured to select, on the basis of the evaluation information by a test item, a user to whom an evaluation result in the past, for a specific test item, is identical to an evaluation result of the first user in the present and to whom the evaluation result has improved from the evaluation result in the past during a period from the past to the present as a candidate for the second user.

8. The health management support device according to claim 1, wherein
the first selection unit is configured to select, as a supported user, a user to whom a classification of evaluation information in the present is “monitoring required”, and
the second selection unit is configured to select, as a goal user, a user having a gender and an age demographic matching those of the supported user, to whom a classification of evaluation information in the past matches the classification of “monitoring required” in the present of the supported user thus selected, and to whom the classification of evaluation information in the present has improved to “normal” or “substantially normal”.

9. A health management support method executed by a health management support device including a storage medium configured to store, in association with each of a plurality of users, information indicating a health state of each user including information indicating a medical examination result, and information indicating a lifestyle of each user, the health management support method comprising the steps of:
selecting, on the basis of the information indicating a health state thus stored, a first user, from among the plurality of users, to be supported in health;
selecting, on the basis of the information indicating a health state thus stored and the information indicating a lifestyle thus stored, a second user, from among the plurality of users, having a health state that is to serve as a goal for the first user thus selected;
reading, from the storage medium, the information indicating a lifestyle of the second user thus selected and generating support information for supporting the health of the first user on the basis of the information indicating a lifestyle thus read; and
outputting the support information thus generated.

10. A non-transitory computer-readable storage medium storing a program for causing a processor to execute processing of each unit included in the health management support device described in claim 1.

11. The health management support device according to claim 2, wherein
the information indicating a health state of each user further includes evaluation information indicating a result of evaluating the information indicating a medical examination result using a plurality of ratings on the basis of criteria defined in advance,
the first selection unit is configured to select, as the first user, a user to whom the evaluation information does not satisfy the criteria set in advance, and
the second selection unit is configured to select a candidate for the second user from among the plurality of users by comparing the evaluation information corresponding to the first user with the evaluation information corresponding to each of the plurality of users.

12. The health management support device according to claim 3, further comprising
a score calculation unit configured to calculate, for each of the plurality of users, a score indicating an effort level in maintaining or improving a health state of each user on the basis of the information indicating a lifestyle of each user thus stored, wherein
the second selection unit is configured to select the second user from among the candidates for the second user thus selected on the basis of the score calculated for each of the candidates.

13. The health management support device according to claim 4, further comprising
a score calculation unit configured to calculate, for each of the plurality of users, a score indicating an effort level in maintaining or improving a health state of each user on the basis of the information indicating a lifestyle of each user thus stored, wherein
the second selection unit is configured to select the second user from among the candidates for the second user thus selected on the basis of the score calculated for each of the candidates.

14. The health management support device according to claim 2, wherein
the information indicating a health state of each user includes
information indicating a medical examination result,
evaluation information by a test item indicating a result of evaluating the information indicating a medical examination result using a plurality of ratings on the basis of criteria defined in advance for each test item, and
comprehensive evaluation information determined on the basis of the evaluation information by a test item,
the first selection unit is configured to select, as the first user, a user to whom the comprehensive evaluation information does not satisfy comprehensive criteria set in advance, and
the second selection unit is configured to select, on the basis of the evaluation information by a test item, a user to whom an evaluation result in the past, for a specific test item, is identical to an evaluation result of the first user in the present and to whom the evaluation result has improved from the evaluation result in the past during a period from the past to the present as a candidate for the second user.

15. A non-transitory computer-readable storage medium storing a program for causing a processor to execute processing of each unit included in the health management support device described in claim 2.

16. A non-transitory computer-readable storage medium storing a program for causing a processor to execute processing of each unit included in the health management support device described in claim 3.

17. A non-transitory computer-readable storage medium storing a program for causing a processor to execute processing of each unit included in the health management support device described in claim 4.

18. A non-transitory computer-readable storage medium storing a program for causing a processor to execute processing of each unit included in the health management support device described in claim 5.

19. A non-transitory computer-readable storage medium storing a program for causing a processor to execute processing of each unit included in the health management support device described in claim 6.

20. A non-transitory computer-readable storage medium storing a program for causing a processor to execute processing of each unit included in the health management support device described in claim 7.
